(19) United States
(12) Patent Application Publication
(10) Pub. No.: US 2003/0158103 A1
(43) Pub. Date: Aug. 21, 2003

(54) Prevention of viral and bacterial disease in shrimp

(75) Inventors: Elliot Entis, W. Newton, MA (US); Kurt Klimpel, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Aqua Bounty Farms, Inc., Waltham, MA

(21) Appl. No.: 10/079,837

(22) Filed: Feb. 19, 2002

(51) Int. Cl. A61K 38/27

(52) U.S. Cl. 514/12

(57) ABSTRACT

It current invention provides methods of increasing resistance of animals of the suborder Natantia to infectious disease. The methods comprise administering growth hormone to the animals in an amount that protects from infection, but does not promote growth.

### BACKGROUND OF THE INVENTION

World demand for shrimp has grown rapidly over the last twenty years. Currently, it is estimated that the global shrimp industry accounts for almost 10 billion dollars annually. Shrimp demand now far outstrips the supply of wild-caught shrimp. Thus, shrimp cultivation, specifically aquaculture farming, has grown significantly to satisfy that demand.

There are a number of problems associated with aquatic farming, however. In particular, aquaculture crops are highly susceptible to disease and pest outbreak. The sources of viral and bacterial infection in aquaculture facilities are varied. Pathogens may enter an aquaculture facility via animal and bird transport. Non-shrimp species, such as crabs, crayfish, squid and other crustaceans, are also sources of infection, as are purchased shrimp stock that contain shrimp infected with disease. Further, shrimp are typically farmed at a high population density in industrial farm operations, which results in stocks that are exceptionally susceptible to disease and infestation. These factors and others can lead to substantial losses for the farms.

To combat this, aquaculture shrimp are routinely dosed with antibiotics and other agents to prevent or reduce infection. However, these treatments have only a limited effect, e.g., antibiotics do not protect against viral infection. Resistance can also emerge, which additionally reduces effectiveness.

Thus, there is a need to develop further strategies to protect aquaculture shrimp species from infectious disease. The current invention addresses this need. The invention is based on the surprising discovery that small doses of growth hormone administered to shrimp at an early age increase shrimp resistance to infection from viral and bacterial agents, thereby decreasing the incidence of disease. As only a modest amount of growth hormone is required to achieve this result, the treatment is also economical and, further, introduces only a small amount of growth hormone into environment.

The application of growth hormone to animals, including aquatic animals, for enhancing growth is well known in the prior art. For example, administration of recombinant bovine growth hormone (rbGH) to 30 juvenile rainbow by intraperitoneal injection and immersion in a growth hormone-containing bath for up to 4 hours accelerated growth. Similarly, administration of recombinant salmon growth hormone (rsGH) to juvenile coho salmon and juvenile chum salmon by immersion also resulted in enhanced growth (see, e.g., Schulte et al, Aquaculture 76:145-156, 1989; and Moriyama et al., Nippon Suisan gakkaishi 56:31-34, 1990).

However, the discovery that growth hormone imparts increased resistance to disease in shrimp has not been previously disclosed. The prior art has only recently disclosed the application of growth hormone to shrimp for the purposes of enhancing growth rates. U.S. Pat. No. 6,238,706 describes the administration of growth hormone via immersion or osmotic shock to various aquatic species, including trout, bass and prawns. The concentrations of growth hormone used for treating shrimp and prawns were much higher than those disclosed in the current application, however, which would represent a substantial economic investment. By contrast, the current invention requires only a small amount of growth hormone and thereby provides an economical means of maintaining the overall health of a population of animals of the suborder Natantia, e.g., shrimp.

### BRIEF SUMMARY OF THE INVENTION

The current invention provides a method of treating members of the suborder Natantia to increase resistance to bacterial and viral pathogens such as white spot virus infection. The method comprises administering growth hormone to the animals in an amount that is effective to reduce the incidence of infection by bacterial and viral pathogens by at least 25% compared to animals that are not exposed to growth hormone. The amount of growth hormone administered is not sufficient to promote growth. The growth hormone can be any growth hormone, such as bovine growth hormone or salmon growth hormone, which are commonly used.

Shrimp species, particularly those that are raised in aquaculture such as P. vannamei, P. styllirostris, or P. monodon., are often treated using the methods of the invention.

In some embodiments, the growth hormone is administered by immersing the animals in a solution containing the growth hormone. Typically, the growth hormone is present at a concentration of between about 0.1 mg/liter and 2.5 mg/liter. Growth hormone is generally administered when the shrimp are between PL 1 and PL 40 in age.

In one embodiment, the animals, e.g., shrimp, are immersed in the growth hormone solution continuously for a length of time while they are in the hatchery, e.g., from 2 to 12 consecutive days, with the concentration of growth hormone constantly maintained.

The method can also comprises a step of dehydrating the animal prior to immersion in the treatment solution. Dehydration can be performed, for example, by osmotic shock, i.e., immersing the animals in a solution having a higher salinity than the animal's normal environment, e.g., about twice the salinity, prior to immersing the animals in the growth-hormone treatment solution, which has a lower salinity than the dehydrating solution. Following dehydration, the animals are typically immersed in the treatment solution for about 3 to about 45 minutes.

In another embodiment, the method comprises administering the growth hormone by feeding the growth hormone to the animals. Typically, the growth hormone is included in the feed in an amount ranging from 1 to 100 g/ton.

In particular, the invention provides a method of decreasing the incidence of a bacterial or viral infection, e.g., white spot baculovirus infection, in young saltwater shrimp, typically P. vannamei, P. styllirostris, or P. monodon, raised in an aquaculture environment. The method comprises immersing the young shrimp aged between PL 1 to PL 40, in a treatment environment comprising salt water and growth hormone in an amount effective to reduce the incidence of bacterial or viral infection by at least 25% compared to young shrimp that are not exposed to growth hormone. The amount of growth hormone used in the method is not sufficient to promoter growth, e.g., between 0.1 mg/liter of saltwater and 2.5 mg/liter of saltwater. The growth hormone is often bovine or salmon growth hormone, and the shrimp are usually immersed in the treatment solution between 2 to 12 consecutive days. This length of exposure is typically not sufficient to promote growth by more than 5%, preferably 1 to 2%. The method can also comprise a step of dehydrating the shrimp prior to immersion in the treatment solution.

In another embodiment, the invention provides a method of increasing disease resistance in saltwater shrimp raised in an aquaculture setting by immersing the shrimp in 0.1 to 10 mg/liter of growth hormone in salt water for at least 5 consecutive days, wherein the exposure is not sufficient to promote growth by more than 1 to 2% and the shrimp are aged between PL 15 to PL 40. The shrimp species treated are usually P. vannamei, P. styllirostris, or P. monodon. Often, the growth hormone is bovine or salmon growth hormone. The method can be used to increase resistance to bacterial and viral infections, such as white spot baculovirus infection.

### DEFINITIONS

The term “growth hormone”, also known as somatotropin in mammals, refers to a hormone secreted by the anterior pituitary. Its main effect is to stimulate growth via binding to its cognate receptor. Included in this definition are growth hormones derived from any animal or plant source, including bovine growth hormone and salmon growth hormone. “Growth hormone” refers to both naturally occurring proteins as well as synthetically produced proteins that share the biological properties of the naturally occurring growth hormones, and which may be identical in structure or which may vary. Thus, “growth hormone” includes nucleic acid and polypeptide polymorphic variants, alleles, mutants, and interspecies homologs as well as active fragments of the peptides.

The amount of growth hormone administered in the subject invention is an amount that is sufficient to provide protection from infection to the animals, but is not sufficient to promote growth. More specifically, treated animals do no experience growth that is greater than 5%, preferably only 1-2%, in excess of the range of average growth for a given untreated species.

The term “saltwater” as used herein refers to an aqueous solution containing a portion of salt (NaCl). The term refers to any quantity of salt in water.

The term “salinity” as used herein refers to the relative proportion of salt in an aqueous solution. Thus, it is a measure of the concentration of salt in a solution. In general it is measured in parts per million (ppm), which indicates the amount in mg/liter; or parts per thousand (ppt), which indicates the amount in g/liter.

The term “seawater” as used herein refers to saltwater having a salinity equal or close to the salinity of ocean saltwater. In general, this salinity is between 28 to 34 ppt.

The term “dehydrate or dehydrating” as used herein refers to diffusion of fluid from an animal such that the fluid content of the animal is lower than its natural condition.

The term “osmosis” as used herein refers to the diffusion of fluid through a semipermeable membrane from a solution with a low solute concentration to a solution with a higher solute concentration until there is an equal concentration of fluid on both sides of the membrane. That solute is most commonly salt. In particular, the term relates to the flow of fluid between an animal's body and the surrounding environment, wherein a salinity gradient exists between the animal and the surrounding environment.

The term “osmotic shock”, as used herein refers to a technique of applying growth hormone to an animal in which the animal is first dehydrated prior to being placed in a rehydrating environment containing growth hormone. The animal then takes up the growth hormone via osmosis as it rehydrates. For example, animals are placed in an aqueous environment which has a higher salinity (usually about 50% to 100% more) than the environment that the animals were in prior to being placed in the high salt environment. The animals are dehydrated by exposing them to the high salt environment for a sufficient amount of time to causes fluids to flow out of the animals body into the surrounding environment via osmosis. In this way the animals are “shocked” osmotically. Subsequently, the animals are placed in an environment containing growth hormone that has a salinity less than the shocking environment, generally its natural environment, such that the animals take up the growth hormone as they rehydrate.

The term “immersion” as used herein refers to placing the subject animals in a tank or other container that contains water and an amount of growth hormone.

The term “PL or PostLarval” as used herein refers to a stage of shrimp development. Shrimp go through a number of stages in their growth cycle. Initially, they pass through a series of larval stages (termed the naupliar, protozoeal and mysis stages) after hatching. This process generally takes 6-9 days and is characterized by rapid growth and development. Afterwards, the shrimp enter the postlarval stage, wherein growth and development is not as rapid but is still significant. The post larval stage lasts between 20 to 50 days, depending on species. Thus, the numerical descriptor associated with PL is roughly the amount of days since the shrimp entered the post larval stage. For example, a PL20 shrimp is a shrimp that has been in the post larval stage for about 20 days and is about 25 to 29 days old from hatching. After the post larval stage the shrimp enter the juvenile stage. Thus, the pre-juvenile stage of shrimp is the larval and post larval stages described above.

The term “aquaculture” as used herein refers to the breeding, raising and harvesting of fresh-and saltwater animals, generally in an artificial environment such as a tank, a pond, or in a enclosed or fenced off portion of the animals natural habitat, such as pond created on the edge of a waterway such as a tidal marsh or lagoon.

The term “incidence of disease” as used herein refers to the occurrence of disease, of any origin, in an animal species population. In this application, this measure is used to compare the number of treated animals in a population that contract a disease against the number of untreated animals in a population that contract a disease, wherein the treated animals have been treated by the methods of the subject invention. Often, the “incidence of disease” is measured by assessing the survival of organisms after infection with a bacterial or viral agent.

The term “promote growth” as used herein refers to the amount of growth of a treated animal that is above or in excess of the growth which would have occurred, on average, in the absence of the application of growth hormone. Growth in an animal species is determined by measuring the increase in weight and/or overall body length over a specified period of time. Often, the growth of an animal can be assessed by comparison to a biometric curve that relates to an animal species growth rate under particular conditions. A small amount of growth, e.g., relative to a biometric curve, may be observed when the animals are treated as described herein to prevent infection. However, shrimp grow rapidly, and this small increase in growth rate is not typically sufficient to substantially alter the length of time required to reach a certain weight, e.g., 25-30 g. The amount of growth hormone applied for the subject invention is that amount which is sufficient to provide protection to the animal but not sufficient to promote growth. More specifically, the treated animals would not experience growth that is greater than 5%, preferably 1-2%, in excess of the average growth for a given untreated species.

The term “protective effect” or “protection” as used herein refers to preventing or reducing the incidence of infection in animals treated using the methods of the invention.

### DETAILED DESCRIPTION OF THE INVENTION

Introduction

The current invention is based on the discovery that immersing animals of the suborder Natantia into an aqueous environment that contains growth hormone is a safe and effective treatment for increasing the resistance of the animals to infectious agents, thus reducing the incidence of disease in the population. In particular, application of a small amount of growth hormone provides protection from disease for these animals.

In general, the invention is applicable to any animal of the suborder Natantia, which include shrimp and prawns, and is most often used to treat shrimp. Usually, the shrimp are raised in an aquaculture or aquafarm. There are over 600 species of shrimp worldwide and the invention can be used to treat any number of these species. However, the methods of the invention are typically applied to those species that are commonly raised as a food source. Suitable shrimps for treatment include members of the genera Penaeus and Lintopenaeus. For example, there are over twelve species of Penaeus, including Penaeus monodon, Penaeus vannamei, Penaeus styllirostris, and Penaeus chiniensis, now being farmed in both Asia and the Americas.

Diseases affecting these animals are well known by those of skill in the art and include both viral and bacterial infections. Among the viruses for which the present invention provides protection from infection are those that are known to cause significant problems in shrimp aquaculture, such as Taura Syndrome Virus (TSV), White Spot Syndrome Virus (WSSV or WSV), or Yellow Head Virus (YHV). Less prevalent diseases include Baculoviral midgut gland necrosis, Nuclear polyhedrosis baculoviroses (Baculovirus penaei and Penaeus monodon—type baculovirus), Infectious hypodermal and hematopoietic necrosis, and Spawner-isolated mortality virus disease. Common bacterial diseases for which the present invention provides protective effects include, but are not limited to, infections by members of the Vibrio genus.

Growth Hormone

The growth hormone to be utilized in the present invention can be any substance, of natural or synthetic origin, which exhibits the biological properties of a natural growth hormone. The natural growth hormone can be of any species, such as bovine, ovine, caprine, equine, porcine, rodent, primate, avian, fish, crustacean, human, and the like.

Growth hormone can be prepared using a variety of techniques. For example, natural growth hormones can be prepared from the pituitary glands of various animals using well-known procedures. Alternatively, the protein can be synthesized in solution or on a solid support in accordance with conventional techniques. Various automatic synthesizers are commercially available and can be used in accordance with known protocols. (See, for example, Stewart & Young, SOLID PHASE PEPTIDE SYNTHESIS, 2D. ED., Pierce Chemical Co., 1984). Further, individual peptides can be joined using chemical ligation to produce the desired end product.

Often, the growth hormone is prepared using recombinant techniques. Recombinant DNA technology can be employed wherein a nucleotide sequence which encodes a growth hormone is inserted into an expression vector, transformed or transfected into an appropriate host cell and cultivated under conditions suitable for expression. The expressed protein is then purified. These procedures are generally known in the art, as described, for example, in Sambrook and Russell, MOLECULAR CLONING, A LABORATORY MANUAL, 3rd Edition, Cold Spring Harbor Press, Cold Spring Harbor, N.Y. (2001); Kriegler, GENE TRANSFER AND EXPRESSION: A LABORATORY MANUAL (1990); CURRENT PROTOCOLS IN MOLECULAR BIOLOGY (Ausubel et al., eds., 1994-1999); and GENE EXPRESSION SYSTEMS, Fernandez & Hoeffler, eds., Academic Press, 1999).

The invention can employ a modified growth hormone, that is, a growth hormone that differs in its structure from a naturally occurring growth hormone, but which retains the biological activity of the known growth hormone and binds to antibodies that bind to a known growth hormone. For example, a modified growth hormone may contain one or more additional amino acids, at one or both ends of the polypeptide chain; may have an amino acid sequence which differs from that of naturally occurring growth hormone; or may be an active fragment of growth hormone. Such modifications to a known growth hormone can include incorporation of amino acid mimetics into the protein. Additional modifications will be understood by those skilled in the art. Therefore, the term “growth hormone” refers to both naturally occurring growth hormones as well as synthetically produced proteins that share the biological properties of the naturally occurring growth hormones, and which may be identical in structure or which may vary.

The amino acid sequences of many suitable growth hormones are known, including human, bovine, ovine, porcine, chicken, rat, mouse, and varieties of fish, e.g., flounder, yellowtail, tuna, salmon (see, e.g., Liu, in Kirk-Othmer “Encyclopedia of Chemical Technology”, 3rd E., Vol. 12, pp. 549-552; Woychik, Nucleic Acid Res. 10:7197, (1982); Liu et al., Arch. Biochem. Biophys., 156:493-508 (1973); Seeburg et al., DNA 2:37-45 (1983); and Watahiki, et al., J. Biol. Chem., 264:3-12 (1989).

Many conservative variations to growth hormone can be used to generate an essentially identical polypeptide. Modifications to growth hormone polypeptide sequences are evaluated by routine screening techniques to ensure that they retain activity. One of skill will recognize that individual substitutions, deletions or additions to a nucleic acid or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

Typically, suitable growth hormone polypeptides have about 50% identity, about 60%, 70%, 75%, 80%, 85%, 90%, or 95-98% amino acid sequence identity to a known growth hormone protein sequence over a comparison window of about 20 amino acids, optionally about 25, 30, or, 50-100 amino acids, or the length of the entire protein. The sequence can be compared and aligned for maximum correspondence over a comparison window, or designated region as measured using one of the following sequence comparison algorithms or by manual alignment and visual inspection. For purposes of this patent, percent amino acid identity is determined by the default parameters of BLAST.

The comparison window includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information.

Cross-Reactivity

Growth hormone for use in the invention typically cross reacts with antibodies to one or more known growth hormone polypeptides. The proteins can therefore be identified based on cross reactivity with antibodies, preferably polyclonal antibodies, that bind to a known growth hormone polypeptide.

Polyclonal antibodies to known growth hormone proteins or fragments thereof are generated using methods well known to those of ordinary skill in the art (see, e.g., Coligan, Current Protocols in Immunology (1991); Harlow & Lane, Antibodies, A Laboratory Manual (1988)). The growth hormone proteins that are immunologically cross-reactive binding proteins can then be detected by a variety of assay methods. For descriptions of various formats and conditions that can be used, see, e.g., Methods in Cell Biology: Antibodies in Cell Biology, volume 37 (Asai, ed. 1993), Coligan, supra, and Harlow & Lane, Antibodies: A Laboratory Manual, Cold Spring Harbor Laboratory, 1988.

Useful immunoassay formats include assays where a sample protein is immobilized to a solid support. For example, a cross-reactive growth hormone protein can be identified using an immunoblot analysis such as a western blot. The western blot technique generally comprises electrophoresing a sample comprising a GH polypeptide on a gel, transferring the separated proteins to a suitable solid support, (such as a nitrocellulose filter, a nylon filter, or derivatized nylon filter), and incubating the sample with antibodies, usually polyclonal antibodies, that bind to known GH polypeptides. The antibodies then specifically bind to cross-reactive GH polypeptides on the solid support. The antibodies may be directly labeled or alternatively may be subsequently detected using labeled antibodies (e.g., labeled sheep anti-mouse antibodies) that specifically bind to the GH antibodies. Other immunoblot assays, such as dot blots, are also useful for identifying GH suitable for use in the invention.

Using this methodology under designated immunoassay conditions, immunologically cross-reactive GH polypeptides that bind to GH antibody at least two times the background or more, typically more than 10 times background, and do not substantially bind in a significant amount to other proteins present in the sample can be identified.

Immunoassays in the competitive binding format can also be used for crossreactivity determinations. For example, polyclonal antisera that have been generated to a known GH polypeptide can be used. The antisera can be immobilized to a solid support. The ability of added GH proteins to compete for binding with known GH polypeptides is analyzed by comparing the binding to a standard curve generated using the known polypeptide. The crossreactivity for the proteins is calculated, using standard calculations.

In order to make this comparison, the standard protein and test protein are each assayed at a wide range of concentrations and the amount of each protein required to inhibit 50% of the binding is determined. If the amount of the test GH protein required to inhibit 50% of binding is less than 10 times the amount of the standard GH protein that is required to inhibit 50% of binding, the test GH protein is said to specifically bind to the polyclonal antibodies.

Formulation and Administration of Growth Hormone

The growth hormone applied to the shrimp may be a single type of growth hormone or may be a combination of growth hormones at various percentages. Further, the growth hormone may be monomeric or dimeric. For example, the growth hormone administered to a subject shrimp species could be 76% monomeric bovine somatotropin, 13% dimeric bovine somatotropin, and 11% aggregated bovine somatotropin. Other additives such as bovine serum albumin may be included but are not preferred. The growth hormone can be prepared in various physical forms. For example, it can be a solution, or can initially be in powder form (e.g. air milled to decrease particle size, granules, etc., such as the milled powder-form bovine growth hormone as taught by EPO 0384752, published Aug. 29, 1990) which is then used to make a solution.

Administration by Immersion

Growth hormone is often administered to the shrimp by immersing the animals into a treatment environment or solution containing water and an amount of growth hormone, for a certain amount of time. There are many methods of doing this. For example, the methods may employ one or more short exposures to growth hormone or may employ continuous exposure.

Typically, the treatment environment water has a salinity approximately equivalent to the animals' natural environment or the environment which they are occupying prior to treatment. The salinity of the water may vary, but is typically at a level that does not affect viability or impede the ability to take up growth hormone. The growth hormone is generally pre-added to the water and is effectively dissolved in the fluid. Thus, the invention is generally practiced by removing the animals from their growing environment, be it the wild or an aquaculture pond or tank, and then placing them into a solution containing growth hormone. The animals are then allowed to bath in the treatment solution for a time sufficient to take up the growth hormone, and are then removed from the treatment solution and placed back in their original environment.

The amount of time the animals are incubated in the growth hormone solution can vary depending on the age of the animals, the concentration of growth hormone, and the type of disease-causing agent or agents for which protection is sought. When the subject invention is practiced as a one-time immersion treatment, i.e., a one-time, short exposure to the treatment solution, the animals are immersed in the growth hormone solution for a length of time that can vary, but is often between 2 minutes to 6 hours, e.g., 30 minutes, 1 hour, 2 hours, or 4 hours. Longer times of exposure can also be used. Shorter times are often used when the growth hormone treatment includes a dehydration step.

Additionally, the animals may be immersed multiple times to increase exposure to the growth hormone and ensure uptake. This can be necessary because a particular species may have difficulty taking up the hormone or may clear the hormone too rapidly from their system. Thus, the present invention may be practiced by immersing the animals in growth hormone multiple times in a single day, or multiple times during one or more weeks of treatment. For example, animals may be immersed multiple times during a week or once a week, for multiple consecutive weeks. The limits to how many times per day or how many consecutive weeks the animals may be treated are conditional, depending on the species of animal. Further, optimum application, in technique or dosage, is partly conditioned on the level of inoculum the animals will be exposed to. As a general rule, if 50% mortality is expected, less exposure and lower doses would be required than if the animals were exposed to levels of inoculum that would produce 100% mortality.

Alternatively, the shrimp may be treated continuously, i.e., the subject animals are allowed to stay immersed in the treatment solution for 1 or more days. The amount of days may vary and can be for as long as 35 or more consecutive days. However, typically the treatment lasts for 7 to 28 consecutive days, e.g., 14 or 21 consecutive days. During continuous immersion, growth hormone is replenished so that the concentration of growth hormone remains constant.

Application of the growth hormone to the subject animals may also be enhanced by dehydrating the animals prior to immersion in the growth hormone solution such that the animals are then rehydrated in the presence of growth hormone. Rehydrating and dehydrating refers to the net flow of water from an animals body, primarily by osmosis. A dehydrated animal would have a lower quantity of water systemically than in its normal state. Upon application of the animal to a rehydrating environment, the animal will uptake water via osmosis until it reaches that normal state. Thus, the animals will more readily take up the growth hormone suspended in the water as it rehydrates. Dehydration of the shrimp may be accomplished by any means known in the art but is preferably accomplished by use of salt gradients.

Dehydrating the subject animals by salt gradients is accomplished by placing the shrimp in an environment that has a higher salt concentration than the salinity of the animals' environment in which they had been growing prior to treatment. The animals respond to the higher salinity by osmotically discharging water from their bodies until a salinity equilibium is reached with the surrounding environment. The dehydrated shrimp are then placed into the treatment environment, which has a salinity lower than the dehydrating environment. The animals will then uptake water (and growth hormone) into their bodies so to once again reach equilibrium with their surrounding environment. In general, the treatment environment will have a salinity approximately equal to the normal or growing environment. For example, a batch of saltwater shrimp is taken from their aquaculture pond having a salinity of seawater and placed in a tank containing additional salt to increase the salinity in order to dehydrate the animal e.g., a salinity that is about twice that of the normal environment. The length of exposure to the high salt can vary, e.g. from 2 to 60 minutes, but is typically from 5 minutes to 40 minutes. The animals are then transferred to a normal saline environment containing growth hormone. The animal then takes up the growth hormone as it rehydrates.

The salinity of the dehydrating solution may vary greatly, but is such that it is sufficient to dehydrate the animal prior to its immersion in the treatment environment. That is, the dehydrating solution may vary according to shrimp species and can be optimized empirically. For example, a salinity that is twice that of the normal environment may be used. Thus, a saltwater species of shrimp that is normally in an environment having a slainity of about 30 ppt can be dehydrated at a salinity of about 60 ppt.

A fresh water shrimp species could be dehydrated in a solution having a salinity that is less than that of seawater, e.g., 10-20 ppt, but which is increased in salinity relative to the fresh water the shrimp is normally maintained in.

The salinity and length of exposure to the dehydrating solution for each species is at a level that does not adversely affect viability.

As noted above, the length of time that the animal is kept in the concentrated salt (dehydrating) solution may vary, but is typically from about 2 minutes to 60 minutes. In general, the greater the difference between the dehydrating environment and the normal growing environment (and the treatment environment), the shorter the dehydration time interval. For example, 15 minutes may be preferable when the dehydrating solution has a salinity approximately 50% higher than the normal growing environment.

Certain species may respond better to certain types of application, e.g., immersion or osmotic shock, than others. Optimization of the administration protocol may be required. Such optimization is readily discoverable by empirical study. In general, continuous application is most preferable with extremely young shrimp and a low concentration of growth hormone.

The concentration of growth hormone in the treatment environment can vary, but is of an amount that does not promote growth that is over 5%, preferably not over 1-2%, of the average of untreated animals. The amount may range between 0.1 mg/liter of water to about 2.5 mg/liter of water. Since growth hormones are not particularly toxic, concentrations above 2.5 mg/liter, e.g. 3 mg/liter or higher, are also possible. Typically, the growth hormone is applied at a concentration between about 0.1 to about 1 mg/liter of water, optimally at about 0.5 mg/L. Fresh water animals are similarly treated but conditions are optimized for such species.

The method detailed herein may be accomplished in any sort of container or environment, including ponds, which can hold the subject animals during treatment. It often is preferable, but not necessary, that the fluid in the environment is fairly static, i.e. the water is not being actively exchanged to an outside environment but instead the system is closed, so as to more readily control the concentration of growth hormone. If there is fluid exchange, growth hormone may be monitored, e.g., by immunoassay, and adjusted on a continual basis. The tanks or ponds may be of any volume.

Other conditions of the treatment environment are, such as the amount of oxygenation, pH, temperature, and the like, are further controlled depending on the duration of the treatment.

The number of animals that are treated in a single environment, i.e., tank, pond, etc., may vary and further depends on the age and the size of the animals treated.

The age of the animals to be treated can also be important in achieving a protective effect. For example, young shrimp develop through several larval stages. The current invention is typically employed when shrimp are at stages of development prior to the juvenile stage (the pre-juvenile stage). This includes the post larval stage as well as naupliar, protozoeal and mysis stages. Usually, shrimp are treated at the post larval stage, PL 1 to PL 40. Preferably, growth hormone is administered to shrimp that are from PL 1 to PL 30 n age, often from PL 1 to PL 12 Thus, while the present invention may be practiced in a pond setting, it is optimally practiced at the hatchery level.

Administration by Feeding

The growth hormone can also be administered to the animals, e.g., shrimp, by feeding. In such embodiments, the growth hormone is preferably formulated in a manner that protects the growth hormone from degradation. A number of such formulations are described in the art. For example, growth hormone can be fed to the animals by including seeds that express growth hormone, e.g., canola seed, in the feed. Such seeds are described, for example in U.S. Pat. No. 6,288,304. Other formulations can include embodiments in which the growth hormone is prepared as an emulsion, e.g., associated with oil-bodies. Such preparations are described, e.g., in U.S. Pat. Nos. 5,948,682; 6,146,645; and 6,210,742.

The amount of growth hormone administered by feeding can vary, but is typically an amount that is 1%, e.g., 0.01%, or less of the feed. Often the amount is 0.005% or less. For example, the amount of growth hormone in the feed can be from about 0.5 grams to 500 grams/ton, and is often from about 1 gram to 100 grams/ton, typically from 5 to 50 grams/ton of feed.

The growth hormone can be administered by feeding at any stage of growth. In some embodiments, the growth hormone is included in feed provided to animals that are cultured in ponds and are typically beyond the hatchery stage, e.g., beyond shrimp that are beyond PL30 or PL40.

The growth hormone can be included in the feed daily, or can be provided periodically, for example, one or more times per week.

Measuring Growth

Growth hormone to increase disease resistance is administered in an amount that does not promote growth. Growth can be measured using techniques known in the art. Typically, growth is assessed by weight and/or length measurements over a specified period of time. In order to determine whether an amount of growth hormone causes growth, comparisons are made of treated animals to untreated animals. Often, the comparisons are made using a biometric curve that are generated specifically for a given environment, such as an aqualculture facility, and reflect the conditions that are particular to that environment.

For example, growth hormone is administered to shrimp in the post larval stage between PL 1 and PL 40. The growth of the animals, as assessed by weight and/or length, during treatment is sampled at specific time intervals and compared to untreated controls. The time intervals can vary. Often, the animals are weighed (and/or the length measured) each day, in particular each day during treatment, or at other regular times, especially during the PL stage. A representative number of treated animals, e.g., 10, can be weighed and compared to control animals. Treated animals can be directly compared to untreated control animals (see, e.g., Example 1) or can be compared using biometric records in the specific hatchery or pond for growth of post-larval shrimp. Lack of significant growth is considered to be growth that is less than 10%, often not more than 5%, preferably not more than 1-2%, in excess of the range of average growth for the untreated animals during the time period measured, which can be, e.g., during the PL stage or during the total length of time the animals are farmed.

### EXAMPLES

The following examples are provided by way of illustration only and not by way of limitation. Those of skill will readily recognize a variety of noncritical parameters which could be changed or modified to yield essentially similar results.

### Example 1

Example 1 illustrates prevention of infection from a viral pathogen in young shrimp treated with growth hormone.

Initially, four treatment environments and four control environments were prepared by filling 8 containers with 25 liters of fresh seawater each, the seawater having an approximate salinity of 32-35 ppt. Recombinant bovine growth hormone (25 mg) was added to each treatment environment so that the treatment environment had a growth hormone concentration of approximately 1 mg/L. About 2500 juvenile P. vannamei shrimp were placed in each of two treatment environments and two of the control environments. About 2500 P. strylirostris (sp. Super shrimp), aged PL 12 were placed in the remaining two treatment environments and control environments. The temperature was controlled with a room heating system and kept nearly constant at 27° C. Water was exchanged at 100% every three days or if the water began to show signs of fouling. The shrimp were fed laboratory diets consistent with their age and size ad lib twice daily.

Growth hormone was added to the environments to keep the concentration of growth hormone at 1 mg/L. Each week, 10 animals were collected at random from each tank and their aggregate weight was determined using an analytical balance.

Following 4 weeks of continuous administration, half of the initial population of shrimp were challenged with viable white spot virus as follows. The shrimp were removed and placed in a separate environment containing 20L of fresh seawater. The animals receiving virus were challenged with 5% biomass of white spot infected tissue for three consecutive days according to established laboratory challenge protocols.

The results are shown in Tables 1 and 2. Table 1 shows that 1.0 mg/L of growth hormone was protective against white spot virus infection. Furthermore, this amount of growth hormone did not stimulate growth (Table 2).

TABLE 1
Survival following 4 weeks of continuous exposure to
1.0 mg/liter growth hormone

| | | Average Time to Death |
|---|---|---|
| Tank | % Survival | (Days) |
| P. vann. (experimental) | 35 | 10.5 |
| P. vann. (control) | 0 | 4 |
| P. stryl. (experimental) | 42 | 11.25 |
| P. stryl (control) | 0 | 3.5 |

TABLE 2
Growth following 4 weeks of continuous exposure to 1.0 mg/liter growth hormone

| | | | | Weight | |
|---|---|---|---|---|---|
| | | | | Difference | |
| | Weight* | Weight* | Weight* | (Aug. 23, 2001 to | |
| Tank | (Aug. 23, 2001) | (Sep. 6, 2001) | (Oct. 4, 2001) | Oct. 4, 2001 | % Increase |
| P. vann. | .3165 | .3694 | .5520 | .2335 | 74.4 |
| (experimental) | | | | | |
| P. vann. | .4217 | .5405 | .7342 | .3125 | 74.1 |
| (control) | | | | | |
| P. stryl. | .0134 | .0202 | .071 | .0576 | 429.0 |
| (experimental.) | | | | | |
| P. stryl | .0135 | .0224 | .072 | .0585 | 433.0 |
| (control) | | | | | |

*Total aggregate weight of ten shrimp collected at random from each tank.

It is understood that the examples and embodiments described herein are for illustrative purposes only and that various modifications or changes in light thereof will be suggested to persons skilled in the art and are to be included within the spirit and purview of this application and scope of appended claims. All publications, patents, and patent applications cited herein are hereby incorporated by reference in their entirety for all purposes.

What is claimed is:

1. A method of increasing resistance to viral and bacterial pathogens in an animal of the suborder Natantia, the method comprising:
administering to the animal an amount of growth hormone effective to reduce the incidence of infection by bacterial and viral pathogens by at least 25% of the amount in the absence of growth hormone;
wherein the amount of growth hormone is not sufficient to promote growth.

2. The method of claim 1, wherein the growth hormone is administered by immersing the animal in a treatment solution comprising water and the growth hormone.

3. The method of claim 1, wherein the animal is shrimp.

4. The method of claim 3, wherein the shrimp are P. vannamei, P. monodon or P. styllirostris.

5. The method of claim 1, wherein the shrimp are aged between PL 1 to PL 40.

6. The method of claim 1, wherein the animals are raised in an aquaculture.

7. The method of claim 2, wherein the animal is dehydrated prior to being immersed in the treatment solution.

8. The method of claim 7, wherein the animals are immersed in the treatment solution for 3 to 45 minutes.

9. The method of claim 2, wherein the animals are immersed in the treatment solution continuously for between 2 to 12 consecutive days.

10. The method of claim 2, wherein the amount of growth hormone dissolved in the treatment solution is between 0.1 mg/liter and 2.5 mg/liter.

11. The method of claim 1, wherein the exposure is not sufficient to promote growth by more than 1 to 2%.

12. The method of claim 1, wherein the growth hormone is bovine growth hormone or salmon growth hormone.

13. The method of claim 1, wherein the infection is caused by white spot virus.

14. The method of claim 1, wherein the step of administering the growth hormone comprises feeding the growth hormone to the animal.

15. The method of claim 14, wherein the animal is shrimp.

16. The method of claim 17, wherein the shrimp are P. vannamei, P. monodon or P. styllirostris.

17. The method of claim 14, wherein the growth hormone is included in the feed in an amount of 0.1% or less.

18. The method of claim 14, wherein the growth hormone is included in the feed in an amount of from 1-100 grams per ton.

19. The method of claim 18, wherein the amount of growth hormone is from 5 to 50 grams per ton.

20. A method of decreasing the incidence of a bacterial or viral infection of young saltwater shrimp raised in an aquaculture environment, said method comprising:
immersing the young shrimp, aged between PL 1 to PL 40, in a treatment environment comprising saltwater and growth hormone in an amount effective to reduce the incidence of the bacterial or viral infection by at least 25% of the amount in absence of said growth hormone; wherein the amount of growth hormone is not sufficient to promote growth.

21. The method of claim 20, wherein the shrimp are P. vannamei, P. monodon or P. styllirostris.

22. The method of claim 20, wherein the amount of growth hormone in the treatment solution is between 0.1 mg/liter and 2.5 mg/liter.

23. The method of claim 20, wherein the animals are immersed in the treatment solution between 2 to 12 consecutive days.

24. The method of claim 20, wherein the animal is dehydrated prior to being immersed in the treatment solution.

25. The method of claim 20, wherein the infection is caused by white spot virus.

26. The method of claim 20, wherein the exposure is not sufficient to promote growth by more than 1 to 2%.

27. A method of increasing disease resistance in saltwater shrimp raised in an aquaculture by immersing the shrimp in 0.1 to 2.5 mg/L of growth hormone in saltwater for at least 5 consecutive days, wherein the exposure is not sufficient to promote growth by more than 1 to 2% and wherein the shrimp are aged between PL 15 to PL 40.

28. The method of claim 27, wherein the shrimp are P. vannamei, P. monodon or P. styllirostris.

29. The method of claim 27, wherein the disease resistance is to white spot baculovirus infection.
